(19) United States
(12) Patent Application Publication
(10) Pub. No.: US 2018/0008182 A1
(43) Pub. Date: Jan. 11, 2018

(54) MULTI-FUNCTION CAPILLARY TUBE SYRINGE WITH RETRACTABLE NEEDLE FOR ARTERIAL BLOOD DRAWING

(71) Applicant: Tony Randall Wann, Kathleen, GA (US)

(72) Inventor: Tony Randall Wann, Kathleen, GA (US)

(21) Appl. No.: 15/206,079

(22) Filed: Jul. 8, 2016

(51) Int. Cl. A61B 5/15; B01L 3/02

(52) U.S. Cl. CPC A61B 5/150534; B01L 3/022; A61B 5/150236; A61B 5/15003; A61B 5/150946; B01L 2300/0838; B01L 2400/0478

(57) ABSTRACT

Implementations of a capillary tube syringe are provided. In some implementations, a capillary tube syringe comprises an elongated hollow outer tube having a proximal end and a distal end and a needle extending from the first end of the outer tube outside the outer tube and extending inside the outer tube. In some implementations, an elongated hollow capillary tube having a proximal end and a distal end extend along the inside of the outer tube. In some implementations, a normally compressed spring is operatively connected to the needle. In some implementations, the spring is configured to expand when activated to retract the needle inside the elongated hollow tube. In some implementations, the capillary tube diameter is less than or equal to 0.865 inches.

### TECHNICAL FIELD

This disclosure relates to implementations of a multi-function capillary tube syringe with a retractable needle for arterial blood drawing.

### BACKGROUND

Drawing blood with disposable syringes remains a common practice that results in a significant number of needlesticks. Existing safety-engineered syringes with retracting features for blood drawing are not specifically designed to draw small amounts (e.g., about 0.5-0.7 cc) of blood. Furthermore, existing safety-engineered syringes with retracting features for blood drawing use traditional plungers.

For example, U.S. Patent Appl. No. 2008/0208119 discloses a safety syringe for injections having a manually retractable exchange needle. The syringe barrel has a plunger slideably located therein. Generally, after an injection has been completed and the needle is removed from the patient, the plunger is pulled back and locks into a needle hub containing the needle and retracts the needle hub and thus the needle into the syringe barrel.

As another example, U.S. Pat. No. 5,800,395 discloses a needle retraction mechanism for a needle bearing medical device. The device comprises a needle carrier structure for retaining a forwardly projecting needle and a rearwardly projecting needle. A re-usable holder adapter is mounted to the rear of the needle carrier structure for receiving a vacuum collection tube. In general, to initiate retraction of the needle, a button is actuated by a user and the collection tube is advanced in a forward direction relative to the barrel by applying pressure to the rear end of the collection tube with the palm of the user's hand. These actions, cause the needle to be retracted by expansion of a biasing spring. Once the needle is in its retracted position, the collection tube can be manually removed.

U.S. Pat. No. 7,918,821 is yet another example of a safety-engineered syringe. The syringe disclosed in the '821 patent is not specifically designed to draw small amounts of blood and also uses traditional plungers.

U.S. Pat. No. 7,803,123 discloses a device for sampling body fluid in the range of less than 50 μL. The '123 patent does not include a means for drawing arterial blood, does not use heparin, and does not include means for separately storing blood. Furthermore, the '123 patent includes a pre-set depth for needle insertion.

### BRIEF DESCRIPTION OF THE DRAWINGS

FIGS. 1-3 illustrate an example capillary tube syringe according to the present disclosure.

### DETAILED DESCRIPTION

Implementations of a capillary tube syringe with a retractable needle for drawing arterial blood are provided. In some implementations, a capillary tube syringe comprises an elongated hollow outer tube having a proximal end and a distal end and a needle extending from the proximal end of the outer tube outside the outer tube and extending inside the outer tube. In some implementations, an elongated hollow capillary tube having a proximal end and a distal end extend along the inside of the outer tube. In some implementations, a normally compressed spring is operatively connected to the needle. In some implementations, the spring is configured to expand when activated to retract the needle inside the elongated hollow tube. In some implementations, the capillary tube diameter is less than or equal to 0.0865 inches.

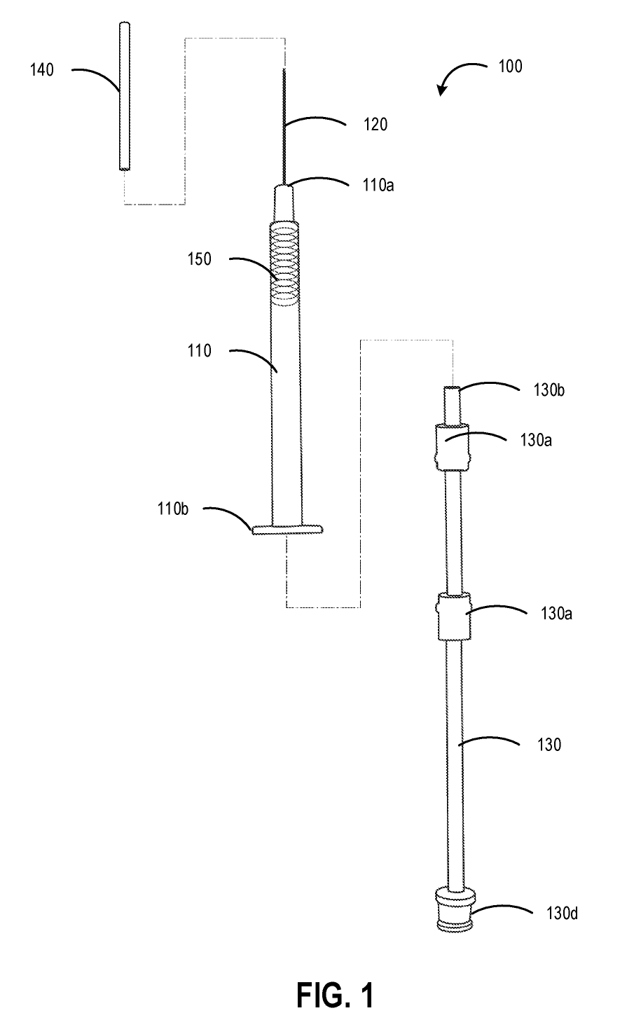

FIG. 1 illustrates an exploded view of example implementation of a capillary tube syringe 100 according to the present disclosure. In some implementations, the capillary tube syringe 100 comprises an elongated hollow tube 110 having a first end 110a for receiving a needle 120 and a second end 110b for receiving a capillary tube 130.

In some implementations, the capillary tube syringe 100 also includes a spring 150 operatively connected to the needle and, in some implementation, operatively connected to the capillary tube 130. In some implementations, the capillary tube syringe 100 may not include a spring.

The capillary tube syringe 100 may also include a cover 140 for enclosing the needle 120. In some implementations the cover 140 may be an elongated hollow tube configured to receive and enclose the needle 120.

In some implementations, the elongated hollow tube 110 is a cylindrical tube. In some implementations, the hollow tube 110 may have a first diameter at a first end 110a and a second diameter at a second end 110b. In some implementations, the first diameter is smaller than the second diameter.

In some implementations, a portion of the tube 110 for receiving the needle 120 may have a diameter of 0.1640 inches or thereabout. In some implementations, the portion of the tube 110 for receiving the needle 120 may have a diameter larger than 0.1640 inches. In some implementations, the portion of the tube 110 for receiving the needle 120 may have a diameter less than 0.1640 inches. In some implementations, the internal diameter of the portion of the tube 110 for receiving the needle 120 may be 0.0865 inches or thereabout to accommodate the cover 140. In some implementations, the internal diameter may be less than 0.0865 inches. In some implementations, the internal diameter may be greater than 0.0865 inches.

In some implementations, a portion of the tube 110 for receiving the capillary tube 130 may have a diameter of 0.26 inches or thereabout. In some implementations, the portion of the tube 110 for receiving the capillary tube 130 may have a diameter larger than 0.26 inches. In some implementations, the portion of the tube 110 for receiving the capillary tube 130 may have a diameter less than 0.26 inches.

In some implementations, the capillary tube 130 may have a diameter of 0.0865 inches or thereabout. In some implementations, the capillary tube 130 may have a diameter greater than 0.0865 inches. In some implementations, the capillary tube 130 may have a diameter less than 0.0865 inches.

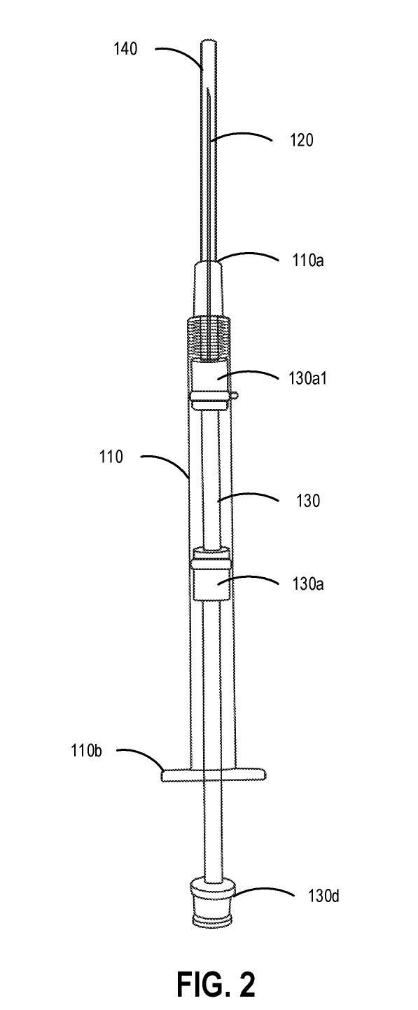

In some implementations, as shown in FIG. 2, one or more capillary tube supports 130a may surround the capillary tube 130 along its length when the capillary tube 130 is positioned inside the hollow tube 110. In some implementations, two capillary tube supports 130a may surround the capillary tube 130. In some implementations, more than two capillary tube supports 130a may surround the capillary tube 130. In some implementations, less than two capillary tube supports 130a may surround the capillary tube 130.

The one or more capillary tube supports 130a may help guide the capillary tube 130 along the hollow tube 110 as the capillary tube is inserted or removed from the hollow tube 110.

In some implementations, a capillary tube support 130a may include an opening through its body to receive the capillary tube 130 thereby surrounding the capillary tube 130.

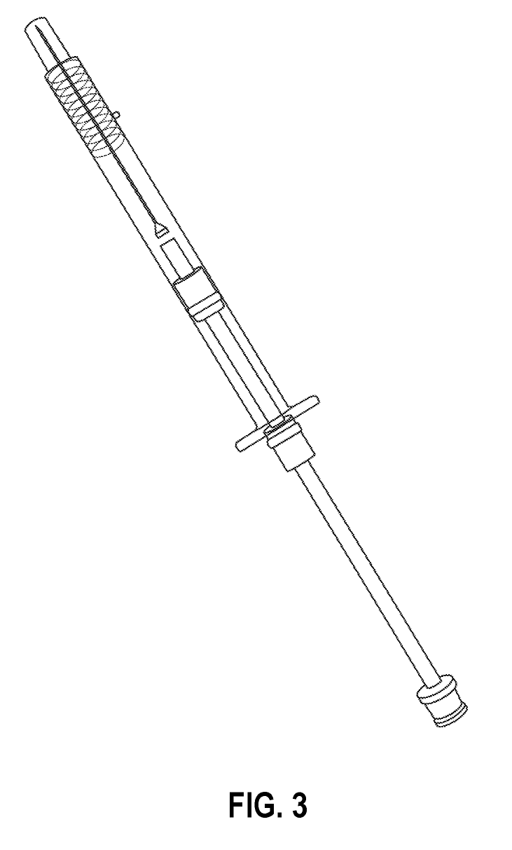

In some implementations, as shown in FIG. 3, the capillary tube supports 130a are sized and configured so that the outer surface of the capillary tube supports 130a may slide along the interior wall of the elongated hollow tube 110 when a pulling or pushing force is applied to the capillary tube 130 to insert or remove a portion of the capillary tube 130 from the tube 110.

In some implementations, the capillary tube supports 130a are sized and configured to remain in place inside the hollow tube 110 to guide the capillary tube 130 as the capillary tube 130 moves from a pulling or pushing force to insert or remove the capillary tube 130 from the tube 110.

In some implementations, the capillary tube 130 is an elongated hollow tube having a first end 130b and a second end 130c. In some implementations, the second end 130c of the capillary tube 130 may include a stopper 130d configured to close the opening at the second end 130c of the capillary tube 130. In this way, blood that is collected in the capillary tube does not leak out the end of the capillary tube.

In some implementations, the capillary tube 130 may be made from plastic. In some implementations, the capillary tube 130 may be made from glass. In some implementations, the capillary tube 130 may be made from any suitable material.

In some implementations, the needle 120 may be a hollow needle. In some implementations, the needle 120 may be a hypodermic needle. In some implementations, the needle 120 may be a 25 gauge needle. In some implementations, the needle 120 may be any suitable gauge needle.

In some implementations, the needle 120 is configured to extend into the first end 130b of the capillary tube 130.

In some implementations, the needle 120 is not configured to extend into the first end 130b of the capillary tube 130. Instead, in some implementations, a gap exists between the needle 120 and the proximal end 130b of the capillary tube 130 (e.g., when the spring is in its normally compressed position). In this way, blood may fill a portion of the inside of the tube 110 and the capillary tube 130. In some implementations the first capillary tube support 130a1 on the proximal end of the tube 110 or the capillary tube 130 may function to stop the blood from entering the lower chamber of the tube 110. In some implementations, the first capillary tube support 130a1 function similar to a plunger tip of a traditional plunger in a traditional syringe to take in and expel blood.

In some implementations, the spring 150 may be a compressed spring that can be expanded, for example, by the press of a button or by the position of the capillary tube 130.

In some implementations, when the spring 150 is expanded, the needle 120 is retracted inside the elongated hollow tube 110 as shown in FIG. 3.

In some implementations, the expansion of the spring 150 also may cause a portion of the capillary tube 130 to be pushed out the second end 110b of the tube 110 as shown in FIG. 3. Although FIG. 3 illustrates the capillary tube supports 130a moving with the capillary tube 130, as discussed above, in some implementations, the capillary tube supports 130a may stay positioned inside the tube 110 as the capillary tube 130 moves.

One of ordinary skill in the art with the benefit of this disclosure would know how to make the capillary tube syringe 100 having a spring 150 as described above.

In some implementations, the capillary tube syringe 100 may be configured to manually retract the needle 120 inside the elongated hollow tube 110. One of ordinary skill in the art with the benefit of this disclosure would know how to make the capillary tube syringe 100 to manually retract the needle 120 inside the elongated hollow tube.

As shown in FIG. 2, when the capillary tube syringe 100 is configured for use, the needle 120 may extend from the first end 110a of the tube 110 and may be operatively connected to the compressed spring 150 inside the tube 110. The portion of the needle extending from the tube 100 may be protected by the cover 140. The capillary tube supports 130a may be positioned inside the tube 110 with the capillary tube 130 extending through the capillary tube supports 130. In some implementations, the second end 130c of the capillary tube 130 may extend from the second end 110b of the tube 110 as shown in FIG. 2. In some implementations, the second end 130c of the capillary tube 130 may not extend from the second end 110b of the tube 110. In some implementations, the second end 130c of the capillary tube 130 may include a stopper 130d. In some implementations, the capillary tube 130 may be operatively connected to the spring 150.

With the capillary tube syringe 100 configured as shown in FIG. 2, for example, in some implementations, to use the capillary tube syringe 100 to collect blood, the cover 140 is removed from the needle 120. The capillary tube syringe 100 may then be used as known in the art. For example, in some implementations, the capillary tube syringe 100 may function as a syringe where the capillary tube 130 is pulled back to extract blood. In some implementations, the capillary tube 130 may remain stationary to extract blood, for example, as in an evacuated tube system. In some implementations, heparin may be used inside the capillary tube 130 and/or tube 110. One of ordinary skill in the art with the benefit of this disclosure will know how to make and use the capillary tube syringe 100 to extract blood using the capillary tube 130 design described above.

Once the blood is collected in the capillary tube 130 (and, in some implementation, in a portion of the tube 110 between the first end 110a of tube 110 and the first capillary tube support 130a1), the needle 120 may be retracted inside the capillary tube syringe 100, for example, by pressing a button or manually. The blood inside the capillary tube 110 (and, in some implementations, the tube 110) may then be tested. For example, the stopper 130d may be removed and the blood may be placed in a blood analyzer. In some implementations, the blood also may be removed from the first end 130b of the capillary tube 110 or the front end 110a of the tube 110.

In some implementations the proximal end of the tube 110 may include threads or other attachments technology or conform to the International Organization for Standardization (ISO) 594 standards (1986 “Conical fittings with a 6% (Luer) taper for syringes, needles and certain other medical equipment) for attaching the capillary tube syringe 100 to an arterial line or another needle (e.g., a larger or smaller needle than needle 120). In some implementations, the needle 120 may be first retracted and then the capillary tube syringe 100 may be attached to an arterial line or another needle.

The above disclosure may be used to perform an arterial stick or a capillary heel stick. For example, the needle 120 may be used for an arterial stick while the distal end of the capillary tube may be used for a capillary heel stick.

Reference throughout this specification to “an embodiment” or “implementation” or words of similar import means that a particular described feature, structure, or characteristic is included in at least one embodiment of the present invention. Thus, the phrase “in an embodiment”, “in some implementations”, or a phrase of similar import in various places throughout this specification does not necessarily refer to the same embodiment.

Many modifications and other embodiments of the inventions set forth herein will come to mind to one skilled in the art to which these inventions pertain having the benefit of the teachings presented in the foregoing descriptions and the associated drawings.

The described features, structures, or characteristics may be combined in any suitable manner in one or more embodiments. In the above description, numerous specific details are provided for a thorough understanding of embodiments of the invention. One skilled in the relevant art will recognize, however, that embodiments of the invention can be practiced without one or more of the specific details, or with other methods, components, materials, etc. In other instances, well-known structures, materials, or operations may not be shown or described in detail.

1. A capillary tube syringe comprising:
an elongated hollow outer tube having a proximal end and a distal end;
a needle extending from the proximal end of the outer tube outside the outer tube and extending inside the outer tube;
an elongated hollow capillary tube having a proximal end and a distal end and extending along the inside of the outer tube; and
a spring operatively connected to the needle wherein the spring is normally compressed and configured to expand when activated to retract the needle inside the elongated hollow tube.

2. The capillary tube syringe of claim 1 wherein the spring is operatively connected to the capillary tube and configured to push a portion of the capillary tube out of the distal end of the outer tube when the compression spring is activated.

3. The capillary tube syringe of claim 1 wherein the capillary tube syringe further comprises one or more capillary tube supports wherein each capillary tube support comprises an opening through its body to receive the capillary tube thereby surrounding the capillary tube and wherein the capillary tube supports are sized so that the outer surface of the capillary tube supports contacts the interior wall of the outer tube.

4. The capillary tube syringe of claim 3 wherein the capillary tube supports are configured to slide along the interior wall of the outer tube when a pulling or pushing force is applied to the capillary tube.

5. The capillary tube syringe of claim 3 wherein the capillary tube supports are configured to remain in place when a pulling or pushing force is applied to the capillary tube.

6. The capillary tube syringe of claim 1 wherein the capillary tube diameter is less than or equal to 0.865 inches.

7. The capillary tube syringe of claim 1 further comprising a stopper positioned on the distal end of the capillary tube and configured to close the opening at the distal end of the capillary tube.

8. The capillary tube syringe of claim 1 wherein a gap exists between the needle and the proximal end of the capillary tube when the spring is in its normally compressed position such that during an arterial blood draw blood fills a portion of the inside of the elongated tube and the capillary tube.

9. The capillary tube syringe of claim 1 wherein the proximal end of the outer tube includes threads configured to attach the syringe to another device.

10. The capillary tube syringe of claim 1 wherein the capillary tube syringe is configured to function as an evacuated tube system such that the capillary tube may remain stationary to extract blood.

11. The capillary tube syringe of claim 1 wherein the capillary tube syringe is configured to function as a syringe such that the capillary tube is pulled back to extract blood.

12. A capillary tube syringe comprising:
an elongated hollow outer tube having a proximal end and a distal end;
a needle extending from the proximal end of the outer tube outside the outer tube and extending inside the outer tube;
an elongated hollow capillary tube having a proximal end and a distal end and extending along the inside of the outer tube; and
one or more capillary tube supports wherein each capillary tube support comprises an opening through its body to receive the capillary tube thereby surrounding the capillary tube and wherein the capillary tube supports are sized so that the outer surface of the capillary tube supports contacts the interior wall of the outer tube.

13. The capillary tube syringe of claim 12 wherein the capillary tube supports are configured to slide along the interior wall of the outer tube when a pulling or pushing force is applied to the capillary tube.

14. The capillary tube syringe of claim 12 wherein the capillary tube supports are configured to remain in place when a pulling or pushing force is applied to the capillary tube.

15. The capillary tube syringe of claim 12 wherein the capillary tube diameter is less than or equal to 0.865 inches.

16. The capillary tube syringe of claim 12 further comprising a stopper positioned on the distal end of the capillary tube and configured to close the opening at the distal end of the capillary tube.

17. The capillary tube syringe of claim 12 wherein a gap exists between the needle and the proximal end of the capillary tube such that during an arterial blood draw blood fills a portion of the inside of the elongated tube and the capillary tube.

18. The capillary tube syringe of claim 12 wherein the proximal end of the outer tube includes threads configured to attach the syringe to another device.

19. The capillary tube syringe of claim 12 wherein the capillary tube syringe is configured to function as an evacuated tube system such that the capillary tube may remain stationary to extract blood.

20. The capillary tube syringe of claim 12 wherein the capillary tube syringe is configured to function as a syringe such that the capillary tube is pulled back to extract blood.
